(19) United States
(12) Patent Application Publication
(10) Pub. No.: US 2008/0200747 A1
(43) Pub. Date: Aug. 21, 2008

(54) Radiopharmaceutical Pigs and Portable Powered Injectors

(76) Inventors: Gary S. Wagner, Independence, KY (US); Frank M. Fago, Mason, OH (US); Keith M. Grispo, Plainfield, IL (US); Chad M. Gibson, Cincinnati, OH (US); John H. Lewis, Lebanon, OH (US); William E. Bausmith, Batavia, OH (US); Elaine E. Haynes, St. Louis, MO (US); David W. Wilson, Loveland, OH (US); Vernon D. Ortenzi, Burlington, KY (US); Elaine Borgemenke, Morrow, OH (US)

Correspondence Address: Mallinckrodt Inc., 675 McDonnell Boulevard, HAZELWOOD, MO, 63042, US

(21) Appl. No.: 11/914,263

(22) PCT Filed: May 16, 2006
(86) PCT No.: PCT/US2006/018727

§ 371 Date: Nov. 13, 2007

Related U.S. Application Data
(60) Provisional application No. 60/681,253, filed on May 16, 2005.
(60) Provisional application No. 60/681,330, filed on May 16, 2005.
(60) Provisional application No. 60/681,254, filed on May 16, 2005.

(51) Int. Cl. A61N 5/00; G21F 5/018; G01N 23/10

(52) U.S. Cl. 600/5; 250/506.1; 422/71

(57) ABSTRACT

One aspect of the present invention is directed to a system for power filling a syringe with a radiopharmaceutical from a vial while attempting to provide low exposure to radiation, and thereafter, power injecting the radiopharmaceutical. A radiation-shielded container of the system generally holds the vial. A filling and injecting device of the system generally includes a mounting structure adapted to support the syringe with a needle of the syringe located in the vial. An electromechanical drive of the system may be commanded by a control to pull a syringe plunger through a controlled motion, thereby filling the syringe.

### CROSS REFERENCE TO RELATED APPLICATIONS

This application is related to and claims the benefit of provisional U.S. Patent Application entitled RADIOPHARMACEUTICAL PIG having Ser. No. 60/681,330 and filed on May 16, 2005; provisional U.S. Patent Application entitled RADIOPHARMACEUTICAL SYRINGE AND PIG COMBINATION having Ser. No. 60/681,254 and filed on May 16, 2005; and provisional U.S. Patent Application entitled RADIOPHARMACEUTICAL FILLING AND DELIVERY SYSTEM having Ser. No. 60/681,253 and filed on May 16, 2005.

### FIELD OF THE INVENTION

The invention relates generally to a powered medical fluid injector and, more specifically, to a powered injector having features such as radiation shielding and/or an energy storage device.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Treatment providers often encounter issues in filling syringes with a radiopharmaceutical on-site. The proper use of radiation shields by technologists during the syringe draw-up and calibration processes is a continuous challenge. Radiation syringe shields for technologists tend to be heavy and awkward to use and may obstruct the view of the radiopharmaceutical as it is being drawn into the syringe. In some situations, the use of syringe radiation shields may impede the handling of the radiopharmaceutical and increase the time spent for the draw-up and dose calibration processes.

Powered injectors are often used in medical settings to inject fluids into a patient. For example, pharmaceuticals are injected into patients with powered injectors during some treatment and diagnostic procedures. Similarly, powered injectors may inject a contrast agent or a tagging agent into a patient. Typically, powered injectors include a syringe and an electric motor to drive the syringe. Generally, the electric motor draws power through a power cord. Unfortunately, the power cord may obstruct movement of the powered injector, thereby potentially rendering the powered injector less convenient to use.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A first aspect of the present invention relates to a radiopharmaceutical pig that facilitates the draw-up of a desired (e.g., correct) unit dose volume of radiopharmaceutical from a container. The radiopharmaceutical pig electronically displays a real-time radioactivity level of a radiopharmaceutical in a container contained in the pig. Therefore, if there is an inventory of several containers of the same radiopharmaceutical, a clinician can quickly, by simple observation of a display on the pig, determine which container is the oldest and should be used first.

Some radiopharmaceutical pigs of the present invention may simplify a determination of a correct unit dose volume by a clinician and thus, reduce the need for a clinician to consult charts, spreadsheets, or use computer programs. Some radiopharmaceutical pigs of the present invention electronically calculate and display the correct unit dose volumes in response to the clinician entering a desired prescription dosage. Certain features of the present invention may be especially useful in manually drawing-up a radiopharmaceutical from a container into a syringe.

A second aspect of the present invention may be said to provide a radiopharmaceutical syringe and pig combination that potentially reduces exposure of persons to radiation from a radiopharmaceutical (e.g., during injection of the radiopharmaceutical into a patient). The radiopharmaceutical syringe and pig combination of this aspect may potentially protect persons from radiation during one or both powered and manual injections of the radiopharmaceutical. Thus, at least some radiopharmaceutical syringe and pig combinations of this aspect may be especially useful in providing protection from radiation during slower, longer duration injections of a radiopharmaceutical.

In a third aspect, the present invention is directed to an apparatus for holding and injecting a radiopharmaceutical. This apparatus includes a pig, a pig cover, and a syringe. The pig has a body that includes one or more appropriate radiation-shielding materials (e.g., lead, tungsten, tungsten-impregnated plastic, etc.). This body of the pig generally has a receptacle defined therein to accommodate at least a portion of the syringe. In addition, this body generally includes an outlet opening that is defined at one end thereof. The cover of the apparatus is designed to be releasably attached to the body to enable a user to cover and uncover the outlet opening on the one end of the body, as desired. The apparatus is designed to support the syringe inside of the body. As such, the syringe remains inside the body of the apparatus during injection of the radiopharmaceutical (from the syringe) to a patient.

With regard to a fourth aspect, the present invention may provide a multi-dose radiopharmaceutical filling and delivery system that permits syringes to be efficiently filled on-site by treatment providers (e.g., at a substantially lesser cost and/or with a substantially lesser risk of radiation exposure). To some, filling and delivery systems of the present invention may tend to reduce risk of radiation exposure during one or both filling of the syringe and injecting the radiopharmaceutical into the patient. To some, the filling and delivery systems of the present invention may reduce risk of radiation exposure during one or both powered and manual injections of the radiopharmaceutical. Accordingly, some embodiments of the filling and delivery systems of the present invention may be especially useful in providing protection from radiation during slower, longer duration injections of a radiopharmaceutical.

In a fifth aspect, the present invention is directed to an apparatus for filling a syringe from a vial containing a radiopharmaceutical. The apparatus generally includes a container that has a base, a cap, and a radiation shield adapted to substantially enclose the vial containing the radiopharmaceutical except for an area of an opening in the base. The apparatus also includes a filling and injecting device that includes a body and a mounting structure. The body of the filling and injecting device generally includes a wall and an opening extending through the wall. The mounting structure of the filling and injecting device is generally adapted to support a syringe, with a needle of the syringe being located in the opening of the body. The container and the filling and injecting device are generally designed so that the wall of the body is capable of receiving the container so that the opening in the base may be positioned immediately adjacent the opening in the body. This arrangement enables the radiopharmaceutical in the vial to be in fluid communication with the needle of the syringe. In at least one regard, this aspect of the invention may be characterized as a power injector and shielding system for radiopharmaceuticals that promotes accurate filling and reduced radiation exposure during filling and injection procedures.

With regard to a sixth aspect, the invention relates to an apparatus for transferring a radiopharmaceutical from a vial having a septum to facilitate in sealing the radiopharmaceutical therein to a syringe. This apparatus includes a filling and injecting device and a container. The container is generally designed to hold the vial in an orientation so that an opening of the container is adjacent the septum of the vial. A radiation shield of the container is generally designed to be substantially disposed about the vial containing the radiopharmaceutical except for an area of the septum. The filling and injecting device of the apparatus includes a body and a mounting structure adapted to support the syringe, with a needle of the syringe located in an opening of the body. The body of the filling and injection devices is designed to receive (or accommodate) at least a portion of the container in a manner so that an opening in the body of the device is immediately adjacent the container opening. Further, the container and device are preferably arranged so that the needle of the syringe pierces the septum, thereby placing the radiopharmaceutical in the vial in fluid communication with the syringe.

In a seventh aspect, the present invention is directed to an apparatus for filling a syringe from a vial containing a radiopharmaceutical. This apparatus includes a container that is adapted to hold the vial containing the radiopharmaceutical and that includes a radiation shield. Further, the apparatus includes a filling and injecting device that includes a mounting structure adapted to support the syringe with the needle of the syringe located in an opening of a body of the device. The body of the device is designed to be disposed about at least a portion of the vial and is generally adapted to place the radiopharmaceutical in the vial in fluid communication with the needle of the syringe. An electromechanical device of the apparatus may be adapted to bias (e.g., push forward and/or draw back) a push rod of the syringe to fill the syringe with the radiopharmaceutical in the vial.

Yet an eighth aspect of the present invention is directed to a method of filling a syringe from a vial having a septum sealing a radiopharmaceutical in the vial. In this method, a container having a radiation shield enclosing a substantial majority of the vial is provided. This container may be said to at least generally hold the vial to locate the septum of the vial adjacent a container opening. A syringe may be disposed in a filling and injecting device to locate a needle of the syringe in an opening of the filling and injecting device. The container may be positioned over the filling and injecting device to locate the septum of the vial over the opening in the filling and injecting device. At least one of the container and the filling and injection device may be moved relative to the other to cause the needle of the syringe to pierce the septum of the vial and place the radiopharmaceutical in the vial in fluid communication with the syringe.

A ninth aspect of the invention is directed to a shielded, cordless injector assembly including an injector, a radiation shield disposed at least partially about the injector, a drive coupled to the injector, and an energy storage device coupled to the drive.

Yet a tenth aspect of the invention is directed to a powered injection system having a syringe, a syringe drive coupled to the syringe, and a capacitor coupled to the syringe drive.

Still an eleventh aspect of the invention is directed to a method in which electrical energy is stored in a cordless injector, and an environment is shielded from a radioactive material within the cordless injector. Further, a flow of the radioactive material is driven with the electrical energy.

Various refinements exist of the features noted above in relation to the various aspects of the present invention. Further features may also be incorporated in these various aspects as well. These refinements and additional features may exist individually or in any combination. For instance, various features discussed below in relation to one or more of the illustrated embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

These and other features, aspects, and advantages of the present invention will become better understood when the following detailed description is read with reference to the accompanying figures in which like characters represent like parts throughout the figures, wherein:

FIG. 1 is a schematic drawing illustrating one embodiment of an improved pig for a container containing a radiopharmaceutical over a life cycle of the improved pig;

FIG. 2 is a perspective view of the improved pig shown in FIG. 1;

FIG. 3 is a schematic block diagram of an electronic circuit implemented in the improved pig shown in FIG. 1;

FIG. 4 is a schematic drawing illustrating further embodiments of a control, dose calibrator and an improved pig for a container containing a radiopharmaceutical;

FIG. 4A is a schematic drawing of an end view of the improved pigs shown in FIG. 4;

FIG. 5 is a schematic drawing illustrating use of a syringe and pig combination;

FIGS. 6A-6C are perspective views of one embodiment of a syringe and pig combination;

FIG. 7A is a schematic cross-sectional view of another syringe and pig combination;

FIG. 7B is a perspective view of the syringe and pig combination of FIG. 6A;

FIG. 7C is a schematic cross-sectional view of still another syringe and pig combination;

FIG. 8 is a schematic drawing illustrating an exemplary embodiment of a multi-dose radiopharmaceutical filling and delivery system;

FIGS. 9 is a cross-sectional view of a vial and vial container mounted on a filling and injecting device used with the multi-dose radiopharmaceutical filling and delivery system of FIG. 8;

FIG. 10 is a front elevation view of the filling and injection device used with the multi-dose radiopharmaceutical filling and delivery system of FIG. 8;

FIG. 11 is a perspective view of a vial container mounted on a filling and injecting device used with the multi-dose radiopharmaceutical filling and delivery system of FIG. 8;

FIG. 12 is a schematic block diagram of a control system of a filling and injecting device used with the multi-dose radiopharmaceutical filling and delivery system of FIG. 8;

FIG. 13 is a schematic block diagram of an exemplary embodiment of a cordless filling and injecting device;

FIG. 14 is a schematic block diagram of an exemplary embodiment of a battery-powered filling and injecting device;

FIG. 15 is a schematic block diagram of an exemplary embodiment of a capacitor-powered filling and injecting device;

FIG. 16 is a diagrammatical representation of an exemplary embodiment of a cordless filling and injecting device and a docking station;

FIG. 17 is a diagrammatical representation of an exemplary embodiment of a cordless filling and injecting device having dual syringes;

FIG. 18 is a diagrammatical representation of an exemplary embodiment of a cordless filling and injecting device having an exemplary syringe;

FIG. 19 is a flowchart illustrating an exemplary embodiment of a nuclear medicine process using one or more of the embodiments illustrated in FIGS. 1-18;

FIG. 20 is a block diagram illustrating an exemplary embodiment of a radio pharmaceutical production system using one or more of the embodiments illustrated in FIGS. 1-18; and

FIG. 21 is a block diagram illustrating an exemplary embodiment of a nuclear imaging system using one or more of the embodiments illustrated in FIGS. 1-18.

### DETAILED DESCRIPTION OF SPECIFIC EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

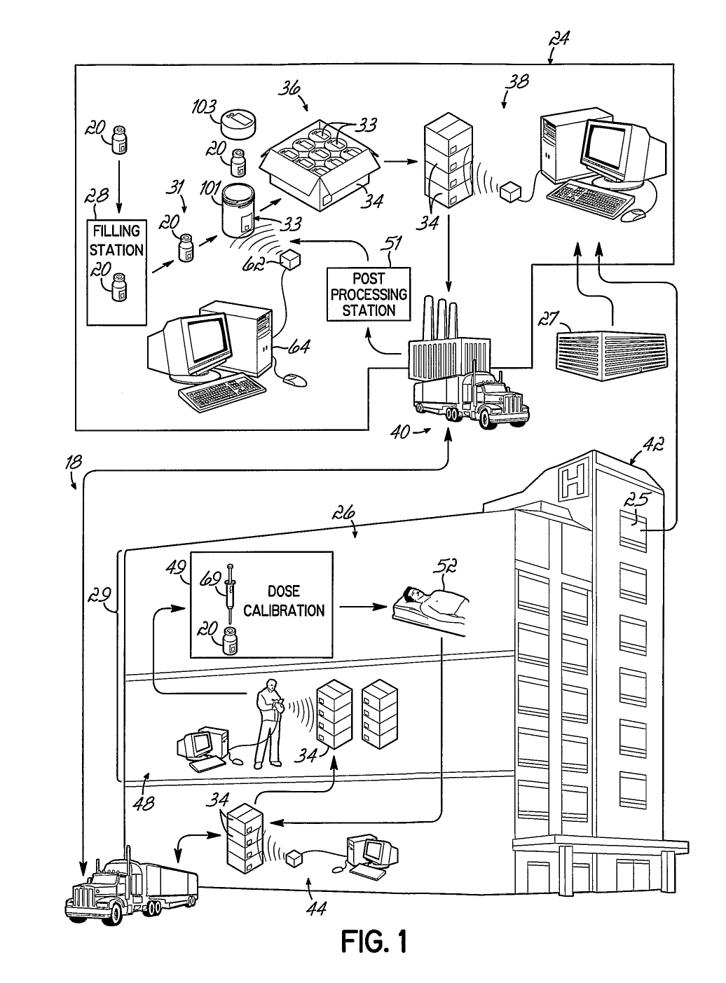

One exemplary life cycle of a radiopharmaceutical container and associated pig is shown in FIG. 1 as a radiopharmaceutical life system 18. Referring to FIG. 1, containers 20 may be filled and packaged at a supplier facility 24 that may or may not be remote from a facility 42 in which the radiopharmaceutical is to be used. Within the supplier facility 24, the container 20 may be filled with a radiopharmaceutical at a filling station 28. A quality control check of the radiopharmaceutical may be performed at quality control station 31; and thereafter, the container 20 may be placed in a pig 33. The loaded pigs 33 may then be packaged either singularly or as a batch in an appropriate shipping carton 34 at a packaging station 36 and the shipping cartons 34 may be temporarily queued or stored in a shipping/receiving department 38.

Orders for the radiopharmaceutical containers 20 can be received from various sources, for example, a purchasing office 25 within a health care facility 42, or a doctor's office 27 that may be part of, or independent from, the facility 42. Further, the orders may or may not be associated with a particular patient. Based on the orders, the shipping cartons 34 may enter a distribution channel 40 by which they may be delivered to a facility 42, for example, a hospital or other health care facility. In the example of FIG. 1, the facility 42 is a hospital that has a shipping/receiving area 44 for receiving the cartons 34 of pigs 33 containing containers 20 filled with radiopharmaceuticals. Often (but not always), the cartons 34 are stored in a nuclear medicine department 29 within the hospital 42, which generally includes a radiopharmacy 48 and/or treatment room 26. As required, a container 20 may be removed from a pig 33; and in a dose calibration process 49, the radiopharmaceutical may be drawn-up from the container 20 into a syringe 69 in preparation for injection into a patient 52.

The correct unit dose volume of radiopharmaceutical to be drawn-up into the syringe 69 generally requires knowing the projected radioactivity level of the radiopharmaceutical at the time the treatment is to be given. To make that determination, it is generally beneficial that one know information such as the radioactivity level at the time the syringe was filled, the filling time and date, the projected treatment time and date, and the rate of decay of the radioactivity of the radiopharmaceutical. Using the projected radioactivity level at the time of treatment and the prescription dosage of the radiopharmaceutical, the correct unit dosage volume can then be determined. Thus, as discussed earlier, the determination of the correct unit dosage volume is difficult and time consuming for a clinician given the tools currently available.

In the described embodiment, the filling station 28, quality control check station 31, container disposal and cleaning of the pig are done at a supplier facility 24 remote from the hospital 42. In an alternative embodiment, one or more of those processes may be done at a radiopharmacy or other location, either within or outside of the hospital.

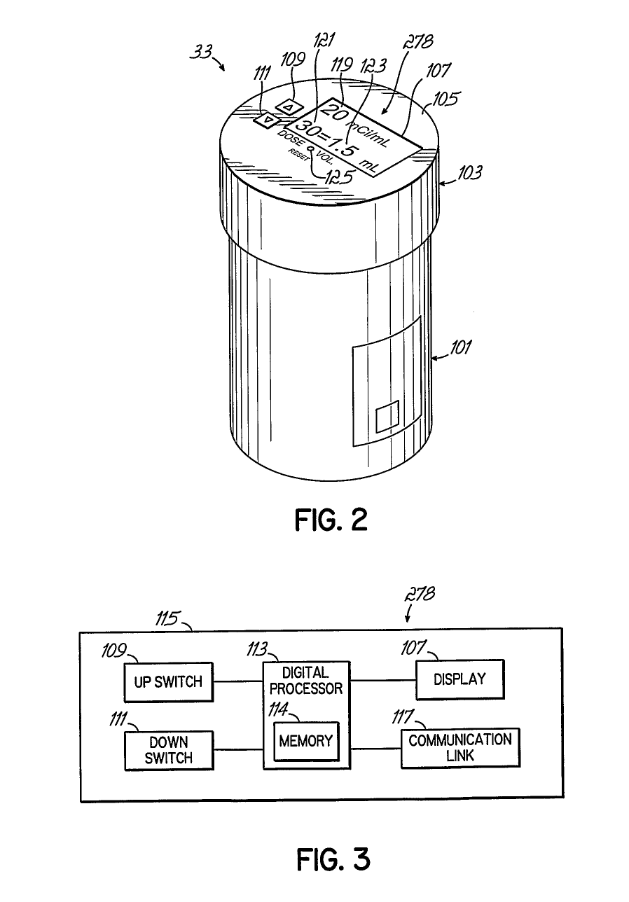

FIG. 2 illustrates a radiopharmaceutical pig 33 that can be used by a clinician to easily determine a correct unit dosage of the radiopharmaceutical. A pig 33 for holding a container containing a radiopharmaceutical has a main body 101 and a lid 103 that is secured to the body in a known manner (e.g., bayonet-type interconnection). The main body 101 and lid 103 may exhibit any appropriate pig design, shape, and construction and is not limited to that illustrated. In other words, the principles of the present invention may be applied to any radiopharmaceutical pig including one or more radiation-shielding materials and used to hold a known syringe or vial.

The lid 103 contains a pig computer 278 that has a display screen 107, an up-switch 109, and a down-switch 111 mounted on a lid upper surface 105. Referring to FIG. 3, the display screen 107, up-switch 109, and down-switch 111 of the pig computer 278 are electrically connected to a digital processor 113 that is mounted with the switches 109, 111 and a display screen 107 on a substrate 115. The substrate 115 is attached to a lid inner surface (not shown) opposite the surface 105 by fasteners, adhesive or other known means. Various other embodiments of the pig 33 may include any of a number of other appropriate locations and arrangements of the display screen 107, up-switch 109, down-switch 111 and/or digital processor 113.

Referring to FIG. 1, within the supplier facility 24, as part of the preparation of the radiopharmaceutical prescription, the pig computer processor 113 may be programmed with data, for example, one or more of an identity and rate of decay of the radiopharmaceutical, a measured radioactivity level of the radiopharmaceutical, a time and date of the measurement, patient's name, projected treatment time and date, the prescription dosage of the radiopharmaceutical, etc. That data may be stored in a memory 114 and can be input to the digital processor 113 via a communications link 117 that may be a wired or wireless link. Further, the data may be entered into the pig computer processor 113 either manually or automatically at a single time or at multiple times (e.g., during the preparation of the radiopharmaceutical prescription).

Knowing the radioactivity level at the time of filling and the rate of radioactive decay, the pig computer processor 113 is designed to automatically update (e.g., in substantially real-time) a radioactivity level of the radiopharmaceutical inside the pig 33. In some embodiments of the pig 33, a current radioactivity level of the radiopharmaceutical inside may be shown on a first numerical display 119 within the display screen 107 with numerical value representing the current radioactivity level in appropriate units (e.g., mCi/mL). Thus, during the period of time that the pig 33 is in storage or transit, the pig computer processor 113 is able to continuously change the numerical value presented by the display 119 to reflect, in substantially real-time, the radioactivity level of the radiopharmaceutical in the container 20. The pig computer processor 113 of some embodiments may also display (in a second numerical display 121 within the display screen 107) a numerical value representing a stored prescription dosage of the radiopharmaceutical. Knowing the real-time radioactivity level and the prescription dosage, the digital processor 113 of such embodiments is able to display (in a third numerical display 123 of the display screen 107) a numerical value representing a correct unit dosage volume of the radiopharmaceutical (e.g., to be drawn into a syringe by the clinician or ejected from a syringe that is already prefilled in the pig).

Immediately prior to injecting the radiopharmaceutical into the patient 52, the clinician may observe the second numerical display 121 representing the earlier programmed prescription dosage of the radiopharmaceutical. If that prescription dosage matches the prescription dosage desired by the clinician, the clinician may then simply read the third numerical display 123 to determine the correct unit dosage volume of the radiopharmaceutical. If the prescription dosage has been changed since the prescription was ordered, the clinician may manipulate the up-switch 109 and/or down-switch 111 to change the numerical value in the second numerical display 121 to match the new prescription dosage.

It may be also desired to change the prescription dosage because the time and date of the treatment have been changed over what was scheduled at the time the prescription was ordered. In that event, the prescribed dosage (e.g., injection volume) of the radiopharmaceutical may be calculated immediately prior to treatment based on the current radioactivity level of the radiopharmaceutical. Within the radiopharmacy 48 of the hospital 42, new values of the radiopharmaceutical radioactivity level and rate of decay and/or prescribed dosage may be entered into the pig computer processor 113 via the switches 109, 111 or the communications link 117 either manually or automatically, for example, using a computer in the calibration tool.

After use, the container 20 may again be placed in the pig 33 and returned to the supplier facility 24. At a post processing station 51, the radiopharmaceutical container 20 may be disposed of; and the pig 33 may be cleaned for reuse (e.g., in a known manner).

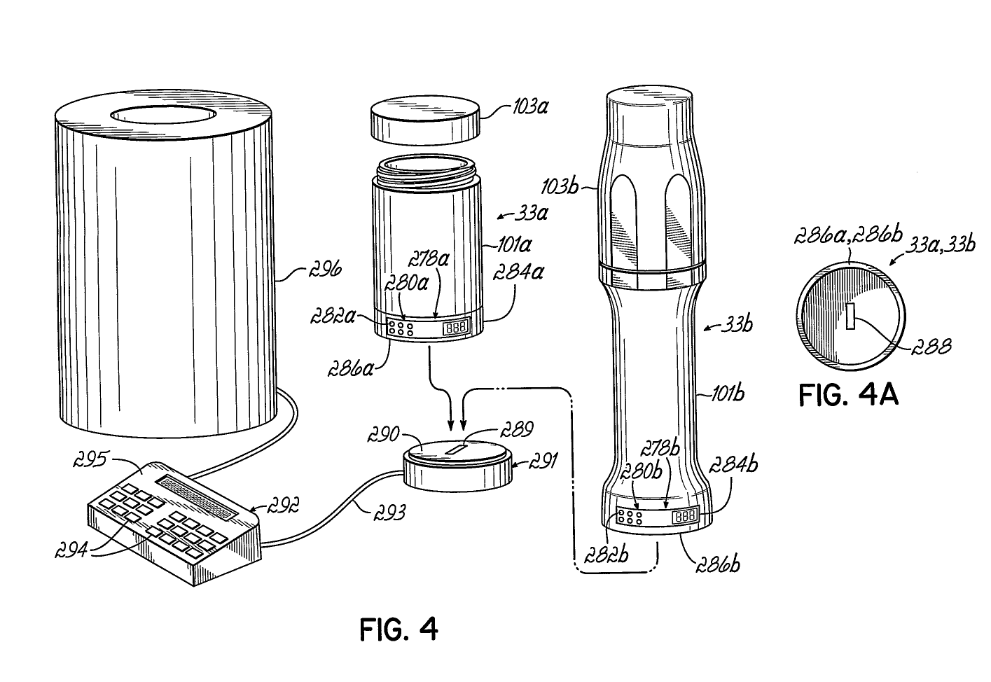

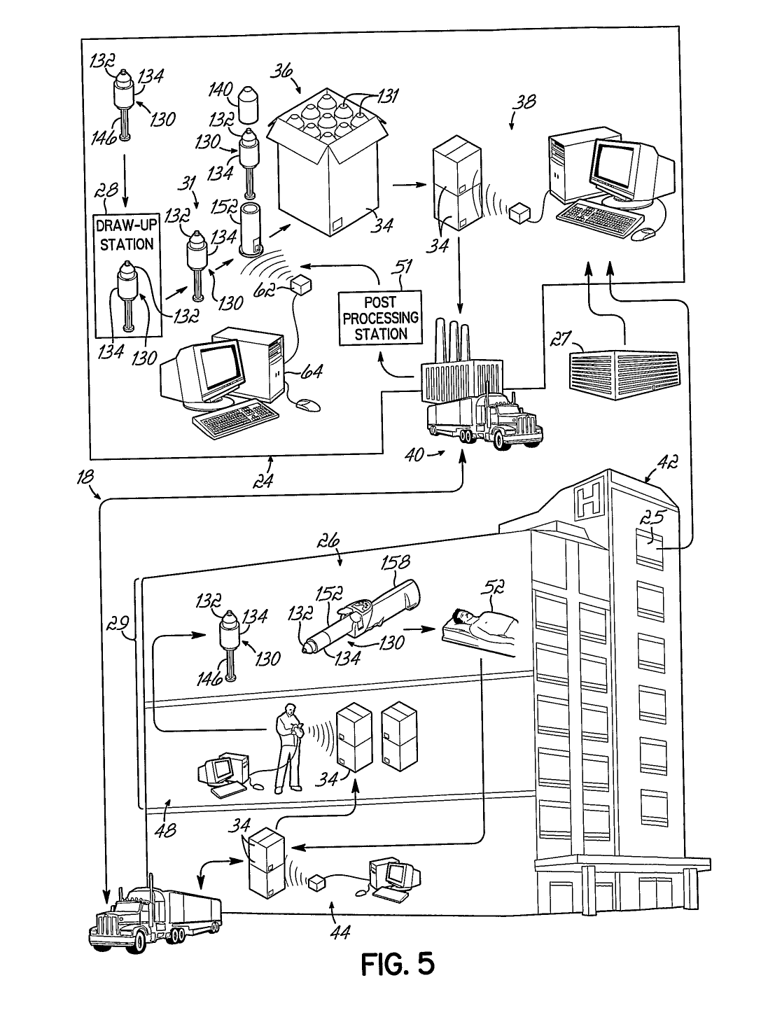

Referring to FIG. 4, further embodiments of a pig containing a microprocessor with an input/output device of some type are illustrated. Pig 33a is designed to hold, store and/or transport a vial containing a radiopharmaceutical; and pig 33b is designed to hold, store and/or transport a syringe containing a radiopharmaceutical. The pigs 33a, 33b have respective main bodies 101a, 101b and respective lids 103a, 103b, which are removable from the respective main bodies 101a, 101b in a known manner for loading and unloading of a radiopharmaceutical vial or syringe.

The pigs 33a, 33b have respective pig computers 278a, 278b that have respective input/output (“I/O”) devices 280a, 280b, for example, respective input switches 282a, 282b and respective output displays 284a, 284b. The input switches 282a, 282b and output displays 284a, 284b are connectable to a pig computer processor in a circuit similar to that shown in FIG. 3. The pig computers 278a, 278b may be used to provide functions substantially similar to the functions described with respect to pig computer 278 of FIGS. 2 and 3. Referring to FIG. 4A, each of the pigs 33a, 33b has a respective electrical connector 288 on respective bottom surface 286a, 286b, which is mechanically connectable to, and provides electrical communication with, an electrical connector 289 mounted on an upper surface 290 of a base unit 291. The electrical connector 289 is electrically connected to a computer in a control unit 292 via a wire connection 293 such as a cable. Therefore, when either of the pigs 33a, 33b is mounted on the base unit 291, thereby mechanically connecting the electrical connectors 288, 289, the respective pig computers 278a, 278b are electrically connected by wires to the computer in the base unit 291.

The control unit 292 has various input devices 294, for example, input keys and/or switches, and output devices 295, for example, a display screen. The control unit 292 is electrically connected to a dose calibrator 296. The dose calibrator 296 has a radiation sensor (not shown) that allows the control unit 292 to monitor the radiation level of a radiopharmaceutical in the dose calibrator in a known manner.

The dose calibrator 296, control unit 292 and base unit 291 are often located in a radiopharmacy and utilized when a radiopharmaceutical prescription is placed in a vial or syringe. The prescribed dosage is put into a vial or syringe using the control unit 292 and dose calibrator 296. Often a label is prepared for application to the vial, syringe and/or pig 33a, 33b, which identifies one or more of the following data: radiopharmaceutical, isotope type, activity level upon being placed in the vial or syringe, predicted dose, patient name, etc. While such data is valuable, the exact time of use can never be known at the time the label is prepared.

However, in the embodiments of FIGS. 3 and 4, the dose calibrator 296, control unit 292, base unit 291, pig processor 113 and input and output devices 284a, 284b, 286a, 286b make up a system that can provide a handler, technician or care giver with a greater quantity of more accurate information relating to dosage of the radiopharmaceutical. In this example, the control unit 292 can transmit to a pig computer processor 113 provided in the vial 33a or syringe 33b data relating to the radiopharmaceutical, isotope type, radiopharmaceutical activity level upon being placed in the vial or syringe, patient name, etc. Further, the pig computer processor 113 can calculate and provide to a respective output device 284a, 284b the time the prescription has been stored in the vial 33a or syringe 33b. Other data can also be determined and displayed, for example, a current real time activity level, a current recommended dosage, etc. Input devices 282a, 282b can be used to retrieve stored data and enter new data, and the display screen 107 may be used to display the data to the clinician. For example, by holding the switches 109, 111 simultaneously depressed for a period of time, the pig computer processor 113 can be programmed to provide an output to the display screen 107 representing an identity of the radiopharmaceutical in the container. In other applications, the switches 109, 111 may be used in a known manner to provide different display options. For example, the display screen 107 may be programmed to turn-off after a period of time to conserve energy; and the display screen 107 can be powered up by holding one of the switches 109, 111 depressed for a period of time. Other switches can be added to provide further display options, for example, a reset switch 125 can be used to reset the operation of the digital processor 113.

With the various embodiments described herein, persons handling the pigs 33a, 33b have up-to-date information relating to the radiopharmaceutical and its age and activity level without having to open the pigs and physically handle the vial or syringe. Thus, potential exposure by handlers to the radiopharmaceutical is reduced. Further, inventories of various radiopharmaceuticals are often maintained; and the output devices 284a, 284b permit a handler to easily determine the oldest pig 33a, 33b, which is often chosen for use.

In the embodiment of FIG. 4, the pigs 33a, 33b are electrically connected to a base unit 291 by electrical connectors 288, 289. In a first alternative embodiment, the base unit 291 and electrical connector 289 may be functionally integrated into the control unit 292. For example, the electrical connector 289 may be mechanically mounted on, and/or integrated into, the control unit 292. Thus, the wire 293 would be internal to the control unit 292 or eliminated if the connector 289 is directly mounted on a printed circuit board or other substrate inside the control unit 292. In other embodiments, the electrical connector 288 may be mounted on an end surface of a pig lid 103a, 103b. In further embodiments, one of the I/O device 280a, 280b and connector 288 may be mounted together on either a respective pig end surface 286a, 286b, or an end surface of a respective lid 103a, 103b. In still further embodiments, a wireless connection can be used, for example, by using a radio frequency identification device (“RF-ID”). An RF-ID system carries data in transponders, generally known as tags; and the data is retrieved by machine-readable means. Thus, an RF-ID tag or transponder having a chip, for example, a programmable processor, associated memory and at least one communications antenna, can be attached to a pig 33a, 33b. Data within the RF-ID chip and associated memory may provide all manner of information relating to a radiopharmaceutical and associated vial or syringe and pig.

An RF-ID system also requires a means for reading data from, and in some applications, writing data to, the tags as well as a means for communicating the data to a computer or information management system. Thus, data is read from, and if applicable, written to, the RF-ID tags by machine-readable means, at a suitable time and place to satisfy a particular application need. Such a machine-readable means can be associated with the base unit 291, or alternatively, with the control unit 292, in which embodiment, the base unit 291 can be eliminated. Thus, an RF-ID system has the versatility to permit data to be written into, and read from, a tag at different times and at different locations.

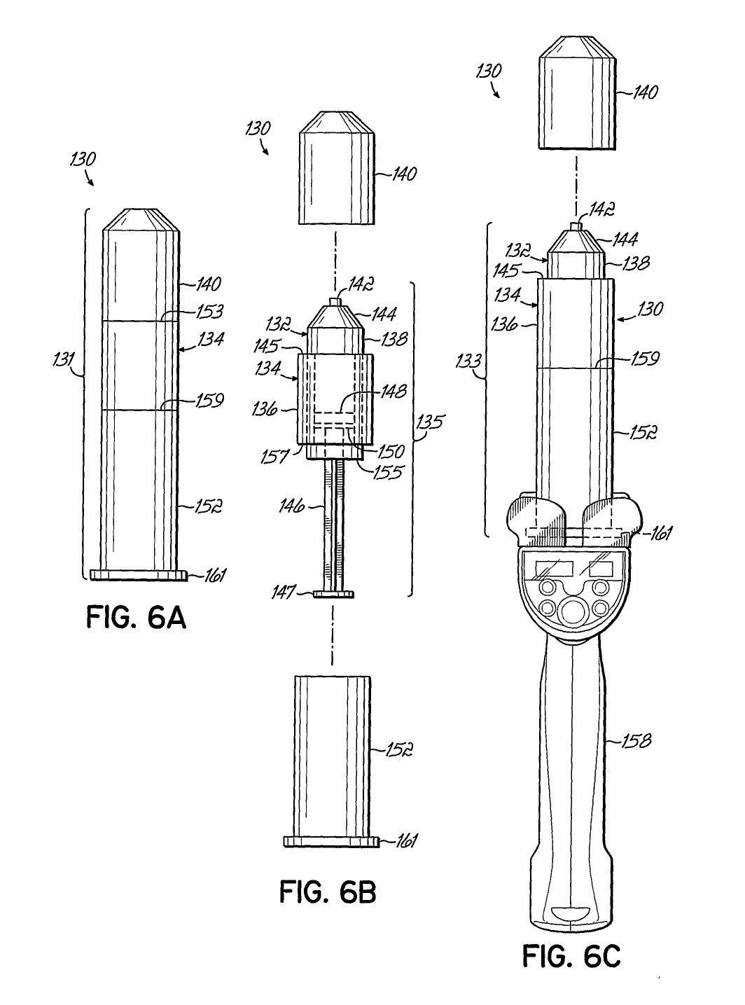

An exemplary life cycle of a radiopharmaceutical syringe and pig combination 130 is shown in FIG. 5. The radiopharmaceutical syringe and pig combination 130 includes a syringe 132 at least generally surrounded by a pig 134. A radiopharmaceutical may be drawn up into the syringe 132 and packaged at a supplier facility 24 that may or may not be remote from a facility 42 in which the radiopharmaceutical is to be used. Within the supplier facility 24, the syringe 132 may be filled with a radiopharmaceutical at a draw up station 28. The pig 134 may or may not be disposed about the syringe 132 during this filling. A quality control check of the radiopharmaceutical may be performed at quality control station 31. Thereafter, outlet end cover or cap 140 and flanged end cap 152 may be attached to the syringe and pig combination 130 to provide what may effectively be characterized as a fully capped syringe and pig combination 131 as shown in FIG. 6A, which may provide a radiation shield from the radiopharmaceutical in the syringe. The capped syringe and pig combination 131 may then be packaged either singularly or as a batch in an appropriate shipping carton 34 at a packaging station 36, and the shipping cartons 34 may be temporarily queued or stored in a shipping/receiving department 38. In a manner similar to that described with respect to FIG. 1, based on orders, the shipping cartons 34 may enter a distribution channel 40 by which they may be delivered to a facility 42 and subsequently provided to a nuclear medicine department 29, which may include a radiopharmacy 48 and/or treatment room 26.

Referring to FIG. 6B, a radiopharmaceutical syringe and pig combination 130 is made up of a syringe 132 and a pig 134. The pig body 136 is mounted over all, or a substantial portion of, a syringe barrel or body 138 and may be wholly or partially made of lead, tungsten and/or any other material that protects persons from exposure to radiation from a pharmaceutical in the syringe 132. The pig body 136 and syringe body 138 may be manufactured as a single integral piece or separate pieces. The pig body 136 may be permanently affixed to the syringe body 138 by a bonding agent, a mechanical connection or may be simply slid over the syringe body 138 in an interference fit. Other manners of disposing a pig about a syringe may be appropriately utilized.

Referring to FIG. 6B, a pig outlet end cap 140 may be used to cover a connector 144 on a syringe outlet end 142. The connector 144 may be sized and shaped to receive tubing. The pig outlet end cap 140 may be mounted to and/or interface with one or both the outlet end 142 of the syringe body 138 or one end 145 of the pig body 136, via an interference fit, a threaded coupling, fasteners or other known means, which provide a joint 153 (FIG. 6A) therebetween that inhibits or even substantially eliminates radiation leakage. The pig outlet end cap 140 may wholly or partially be made of lead, tungsten and/or any other material that protects persons from exposure to radiation from a pharmaceutical in the syringe 132.

The syringe 132 includes a plunger rod 146 that extends into the syringe body 138 and is connected to a plunger 148. The plunger rod 146 has an outer end 147 that may be made to any desired size and shape to interface with a translatable drive shaft (not shown) inside the injector 158 (FIG. 6C), so that the injector drive shaft can push the plunger 146. The plunger 148 may be wholly or partially made of lead, tungsten and/or other material that shields persons from exposure to radiation from the pharmaceutical in the syringe 132. In the exemplary embodiment of FIG. 6B, the plunger 148 has a radiation shield layer 150. Thus, the pig body 136 and radiation shield layer 150 on the plunger 148 provide some radiation protection near the plunger rod 146.

The pig 134 has a flanged end cap 152 that is removably attachable to either an opposite end 155 of the syringe body 138, or an opposite end 157 of the pig body 136, via an interference fit, a threaded coupling, fasteners or other known means, which provide a joint 159 (FIG. 2A) therebetween that inhibits or even substantially eliminates radiation leakage. The pig flanged end cap 152 may be wholly or partially made of lead, tungsten and/or any other material that protects persons from exposure to radiation from a pharmaceutical in the syringe 132.

The fully capped pig and syringe combination 131 (FIG. 6A) can be used to inject the radiopharmaceutical either manually or with a power injector. For manual injection as shown in FIG. 6B, the pig end caps 140 and 152 may be removed to provide a fully uncapped pig and syringe combination 135. Tubing (or other appropriate delivery conduit) may be connected to the connector 144. A clinician may then depress the plunger rod 146 to inject the radiopharmaceutical. To use with a power injector, only the end cap 140 may be removed; and, as shown in FIG. 6C, the flanged end cap 152 may remain attached to provide a partially capped syringe and pig combination 133. The flanged end cap 152 may be designed to permit the syringe and pig combination 130 to be mounted in a power injector 158. An exemplary power injector that may be suitable for use with the partially capped syringe and pig combination 133 is shown and described in U.S. Patent Application Publication No. US 2004/0024361 A1 entitled “Injector” and assigned to the assignee of the present application. The entirety of U.S. Patent Application Publication No. US 2004/0024361 A1 is hereby incorporated by reference herein.

When used either manually or with a power injector, the presence of the radiation shields provided by the pig body 136, the plunger layer 150, if used, and flanged end cap, if used, may be said to at least generally inhibit radiation exposure to persons administering the radiopharmaceutical. After ejection of the radiopharmaceutical from the syringe 132, the end caps 140 and 152 may be attached as shown in FIG. 6A, and the fully capped syringe and pig combination 131 may be returned to the supplier facility 24. At a post processing station 51, the syringe may be disposed and the pig 134 and end caps 140, 152 may be cleaned for reuse.

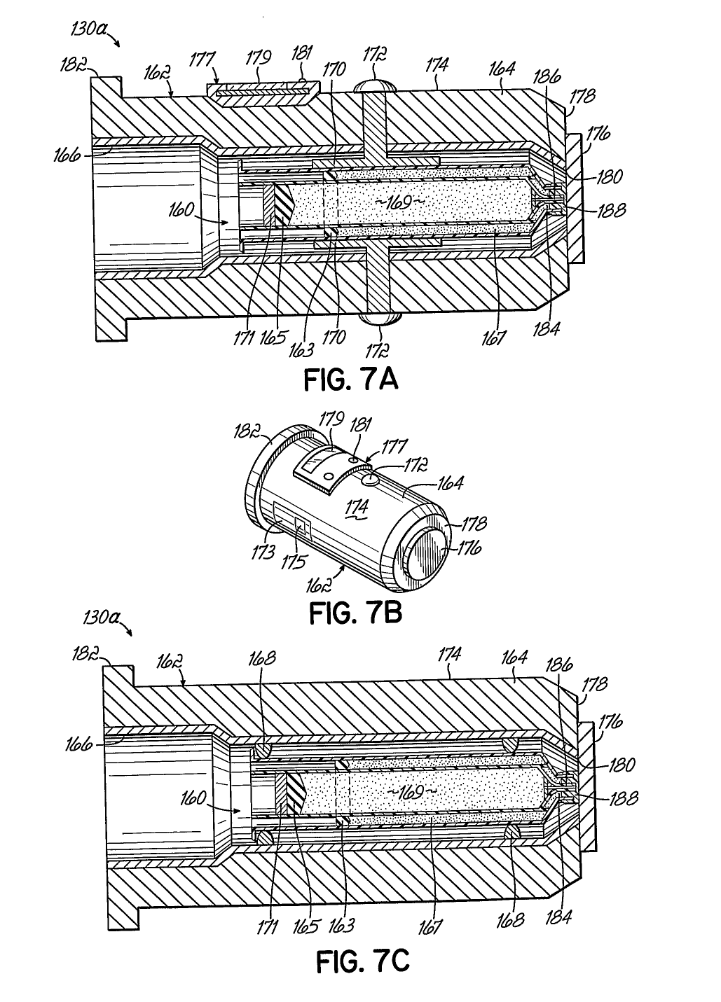

Referring to FIG. 7A, another embodiment of a syringe pig combination 130a includes a syringe 160 mounted within a pig 162. The pig 162 has an outer covering 164 of lead, tungsten and/or other radiation shield material and an internal liner 166. In this embodiment, the syringe is a two-stage syringe having first and second plungers 163, 165 respectively. The syringe 160 has a first cavity 167 with a first outlet end 184 and a second cavity 169 with a second outlet end 186. A tip 188 is removably mounted over the outlet ends 184, 186. The first cavity 167 may be filled with a radiopharmaceutical, and the second cavity 169 may be filled with a saline solution and/or other appropriate biocompatible flush (e.g., heparin solution, sterilized water, glucose solution, etc.). The syringe 160 may be secured in the pig 162 by any suitable means (e.g., by retractable grippers 170 that are biased against an outer surface of the syringe 160). The grippers may be released by actuating a release button 172 mounted on an outer surface 174 of the pig 162.

The first plunger 163 is torroidally shaped and contacts cylindrical walls forming the outer, first cavity 167, and the second plunger 165 is shaped to fit inside of, and contact the cylindrical wall forming, the inner, second cavity 169. The plungers 163, 165 may wholly or partially be made of lead, tungsten and/or other radiation shield material. In the exemplary embodiment of FIG. 7A, the plunger 163 is made wholly of a radiation shield material, whereas the plunger 165 has an outer directed layer 171 of radiation shield material. Further, in the exemplary embodiment of FIG. 7A, the pig 162 may be sized and shaped to correspond to a syringe of a standard size (e.g., 125 milliliter syringe) and may have a flange 182 that permits the syringe and pig combination 130a to be mounted in an injector. Examples of manual and power Injectors suitable for operating a dual cavity or two stage syringe 160 are shown and described in U.S. Provisional Application No. 60/695,467, entitled “Dual Chamber Syringe”, filed Jun. 30, 2005 and assigned to the assignee of the present application. The entirety of U.S. Provisional Application No. 60/695,467 is hereby incorporated by reference herein.

An end cover 176 may be mounted on a pig end surface 178 over a pig outlet opening 180. The cover 176 may be made from lead, tungsten and/or other material providing a radiation shield from the radiopharmaceutical. The cover 176 can be designed to slide or fit over the surface 178 to selectively uncover and cover the opening 180. Alternatively, the cover 176 can be pivotally mounted on the surface 178 to enable a user to selectively uncover and cover the opening 180 as desired. As a further alternative, the cover 176 can be secured to the end surface 178 by removable fasteners, thereby permitting a user to cover and uncover the opening 180 as desired. Incidentally, other manners of providing a cover and uncover feature are contemplated as well as combinations of the various possibilities described above.

As shown in FIG. 7B, the pig 162 may have a printed label 173. Further, the pig 162 may have a radio frequency identification device (“RF-ID”) 175 that may be part of, or independent of, the label 173. Data relating to the radiopharmaceutical, the syringe 160 and the pig 162 can be read from and/or written to the RF-ID at every stage of respective life cycles of those components. In addition, the pig 162 may have a user interface 177 including a display screen 179 and/or input devices 181, for example, switches, mounted on the outer surface 174. The display screen 179 and/or switches 181 may be connected to a digital processor (not shown) having a memory that may be used to store data relating to the radiopharmaceutical, its radioactivity level, etc.

The continued presence of the radiation shields provided by the pig 162 and the plunger layer 171, if used, during an injection of the radiopharmaceutical into a patient, may be said to at least generally inhibit radiation exposure to persons handling the syringe and pig combination 130a and administering the radiopharmaceutical.

In an alternative embodiment shown in FIG. 7C, a syringe 160 can be secured within the pig 162 by means of annular (or other appropriately designed) projections 168 that provide an interference fit of the syringe 160 inside the pig 162. In further embodiments, depending on the radioactivity level of the radiopharmaceutical, the radiation shield protection of the plungers may be eliminated. Further, depending on the level of radioactivity of the radiopharmaceutical, the flanged end cap 152 may be made of a material that does not provide a radiation protection shield.

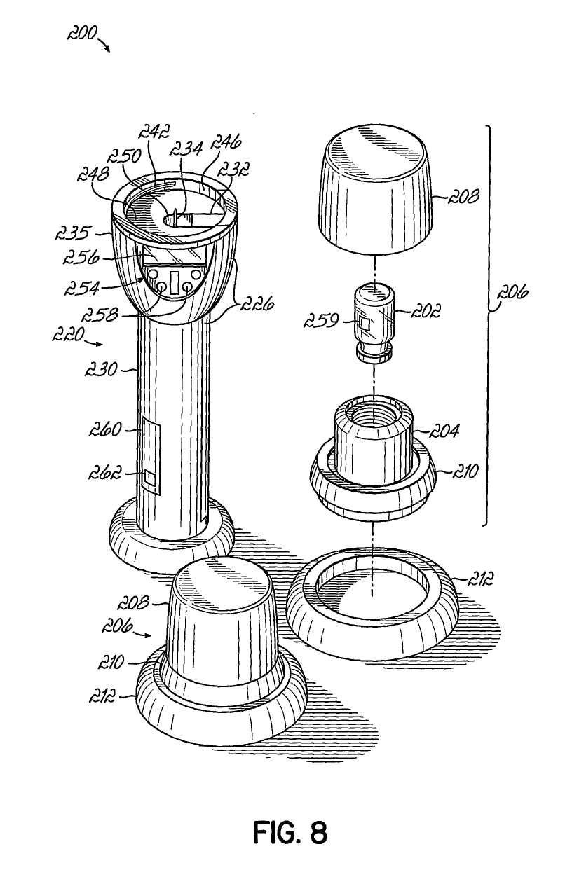

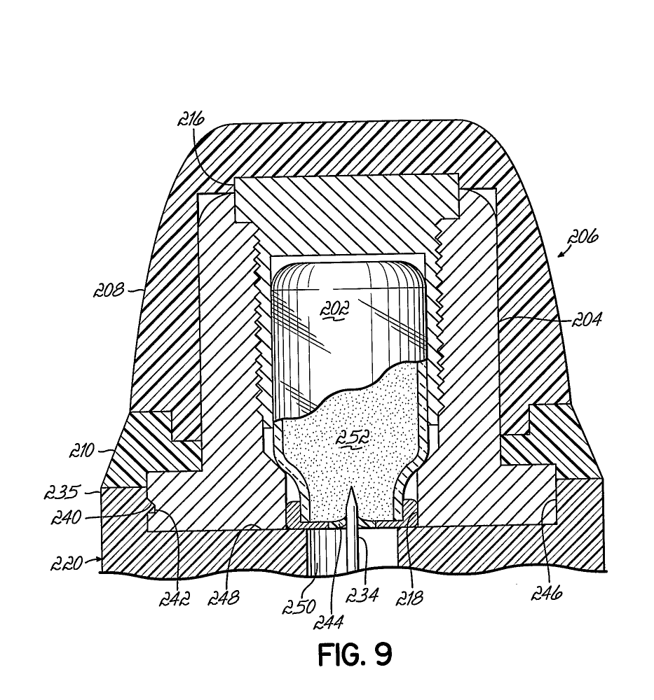

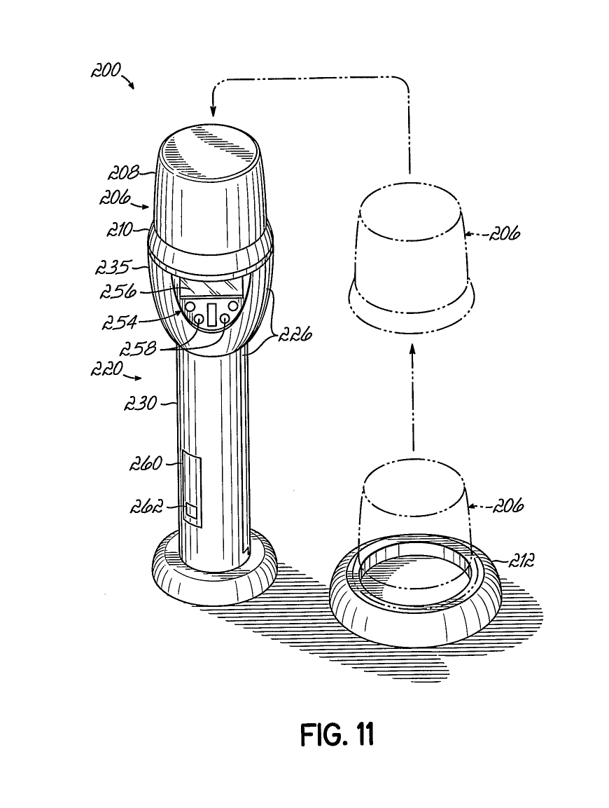

The various components of an exemplary multi-dose radiopharmaceutical filling and delivery system 200 are shown in FIG. 8. This filling and deliver system 200 may be suitable for use at a site of a treatment provider. The filling and delivery system 200 generally includes a shielded radiopharmaceutical container 206 and a filling and injecting device 220. Incidentally, the radiation shielding of the container 206 may be any appropriate shielding material (e.g., lead, tungsten plastic, and/or tungsten). As will be described, after disposing a syringe in the filling and injecting device 220, the radiopharmaceutical container 206 may be mounted on top of the filling and injecting device 220 as shown in FIG. 11. The filling and injecting device 220 may then be operated to provide what may be characterized as a powered filling of the syringe with a prescribed unit dose volume of a radiopharmaceutical. The powered filling process of some embodiments may be characterized as fast, accurate, and/or presenting less risk of exposure to radiation than known systems. Thereafter, the container 206 may be dissociated from the device 220, and the filling and injecting device 220 may be operated to provide a power injection of the radiopharmaceutical into a patient. Alternatively, the syringe can be removed from the filling and injecting device 220 and used manually to inject the radiopharmaceutical into a patient.

The treatment provider purchases from a pharmacy a radiopharmaceutical in a multi-dose vial 202 (FIG. 8), and the vial 202 may be removed from its shipping pig and placed inside a vial holder 204 of a container 206. The vial holder 204 may be fixed to a container base 210, and a container cap 208 may be secured over the container base 210. As shown in FIG. 9, a threaded plug 216 may be mounted inside the cap 208; and thus, the cap 208 may be firmly secured to the base 210 by threadedly engaging the threaded plug 216 with internal threads on the holder 204. The mechanical connection between the cap 208 and base 210 may be any appropriate interconnection such as a quick turn thread (e.g., a high helix thread, a bayonet style thread, etc.). The vial holder 208 and plug 216 may include any appropriate radiation-shielding material (e.g., lead, tungsten plastic, and/or tungsten) capable of providing radioactive shielding from the radiopharmaceutical within the vial 202.

The container 206 at least generally permits the radiopharmaceutical vial 202 to be conveniently handled and carried while providing radiation protection about the vial 202 (e.g., except at the opening 218). Incidentally, nuclear medicine department personnel are used to handling devices having radiopharmaceuticals disposed therein that have a “live” or “hot” opening, and so, the opening 218 does not represent a new handling discipline. To cover the opening 218, the container 206 may be placed in a base support or coaster 212 that includes any appropriate radiation-shielding material. Thus, when placed on the coaster 212, the radiopharmaceutical within the container 206 is substantially shielded. The container 206, cap 208, and/or coaster 212 can be patterned, labeled, and/or color coded to permit a quick visual identification of different radiopharmaceuticals or other predetermined designations.

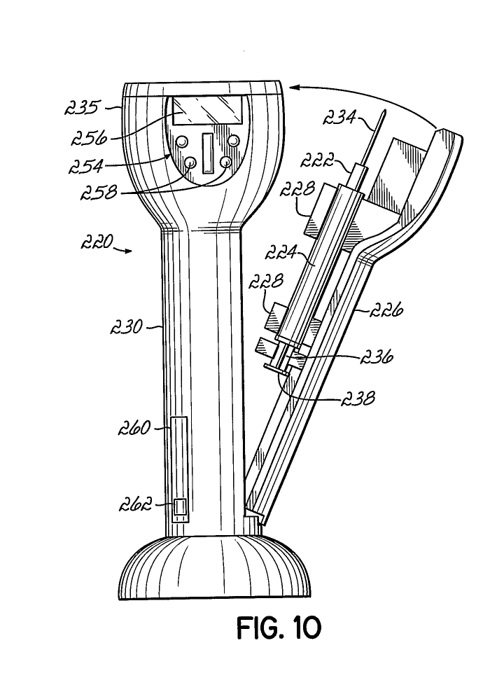

The filling and delivery system 200 further includes a filling and injecting device 220 shown in FIG. 10 that provides a powered filling or dispensing of a radiopharmaceutical from a syringe 222 supported therein. Prior to being mounted in the filling and injecting device 220, the syringe 222 may be inserted into a syringe radiation shield 224. The radiation shield 224 may have one or more internal projections and/or other appropriate device(s) to at least assist in securing the syringe 222 in the shield 224 so that the shield 224 and syringe 222 do not separate during normal handling, but so the syringe 222 can be separated from the shield 224 when desired. The radiation shield 224 may be said to provide a first level of radiation shielding as the syringe 222 is manually manipulated, handled and/or used directly to inject a radiopharmaceutical into a patient. The syringe radiation shield 224 may exhibit a standardized external size and/or shape to facilitate securing the syringe 222 in the proper orientation within the filling and injecting device 220. Thus, syringes of different sizes may be held with mating syringe shields that all may have a common or similar exterior size and/or shape. The shielded syringe holder 224 may be made of tungsten, tungsten plastic, lead and/or any other material that provides a radiation shield from the radiopharmaceutical. Further, the shape and size of the shielded syringe holder 224 may vary (e.g., to meet functionality, ergonomic and/or shielding requirements of different applications).

In the exemplary embodiment of FIG. 10, the filling and injecting device 220 has a removable side wall 226 with a pair of U-shaped resilient clamps 228 that secure the syringe shield 224 at a desired position and orientation. After properly locating the shielded syringe holder 224 in the clamps 228, the side wall 226 may be then repositioned against a body 230 of the filling and injecting device 220. A syringe needle 234 may be moved through a slot 232 (FIG. 8) in an upper wall 248 of the filling and injecting device 220 and may be is located in a centerhole 250. The syringe needle 234 preferably extends through and above the upper wall 248 as also shown in FIG. 9.

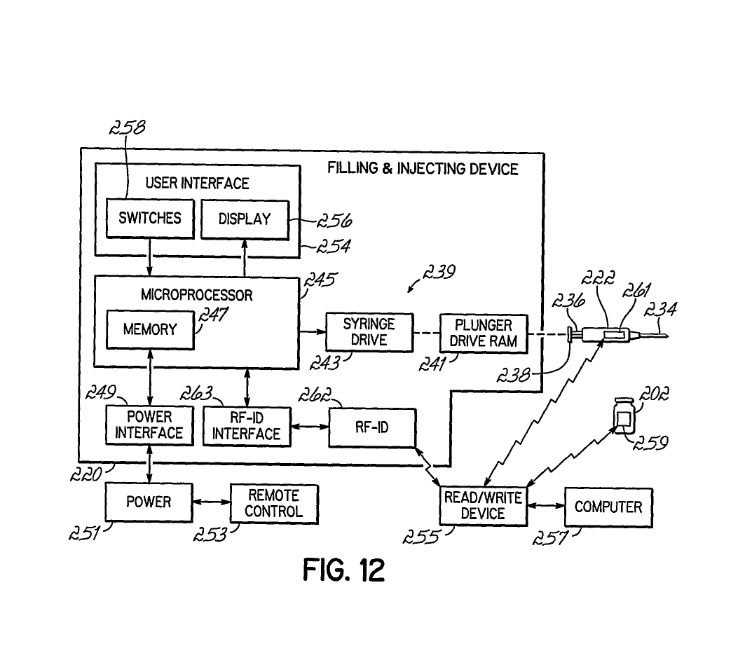

As shown in FIG. 12, the syringe 222 may have a push rod 236 with a flanged end 238. The flanged end 238 may be sized and shaped to interconnect with an end of a powered translatable electromechanical drive 239 housed in the filling and injecting device 220. The electromechanical drive 239 is shown as including a plunger drive ram 241 connected to a syringe drive 243, for example, an electric motor. The operation of the syringe drive 243 may be controlled by a microprocessor 245 having a memory 247. The microprocessor 245 may be connected to a power interface 249 that, in turn, is connected to a power supply 251. The microprocessor 245 may further be connected to a user interface 254 (FIG. 8); and the user interface 254 may include but is not limited to a display screen 256 and/or input devices 258, for example, switches. The memory 247 may be used to store data relating to the operation of the filling and injecting device 220, which may include but is not limited to a program to control filling and/or injecting operations, information relating to the radiopharmaceutical, other procedural or non-procedural information, patient information, provide communication back to pharmacy, etc. A remote control 253 may be utilized to assist in providing communication to the pharmacy, manufacturer, etc. The display screen 256 may incorporate alphanumeric and/or graphic displays to display data that includes but is not limited to filling and/or injecting parameters, status, installed components, radiopharmaceutical information, instructions, warnings, etc. A remote control 253 connected to the power supply 251 may optionally be used instead of the user interface 254 for remote operation of the filling and injecting device 220.

A control and electromechanical drive of the type illustrated in FIG. 12 and that may be suitable for use in the filling and injecting device 220 is shown and described in U.S. Patent Application Publication No. US 2004/0024361 A1 entitled “Injector” and assigned to the assignee of the present application; and the entirety of U.S. Patent Application Publication No. US 2004/0024361 A1 is hereby incorporated by reference herein.

As shown in phantom in FIG. 11, the container 206 may be lifted off of the coaster 212 and placed over the upper end 235 of the injecting and filling device 220. As shown in FIG. 9, the container base 210 with its radioactive shield 204 is located in a cavity 246. The container 206 and filling and injection device 220 may be engaged by contracting the threads 240 with the threads 242 and subsequently rotating one with respect to the other, thereby engaging the threads 240, 242. Engagement of the threads 240, 242 translates the container 206 with respect to the filling and injecting device 220, and a septum 244 on a lower end of the vial 202 may be pierced by the needle 234 extending through the upper wall opening 250. Thus, the needle 234 may be placed in fluid communication with the radiopharmaceutical 252 in the vial 202. Upon the container 206 and the filling and injecting device 220 being fully secured together, the septum 244 is generally located immediately adjacent the upper wall 248.

In alternative embodiments, the mechanical connection between the container 206 and filling and injecting device 220 may be any quick turn thread or any other quick connect and disconnect device. In another embodiment, the mechanical connection, for example, the threads 240, 242, can be eliminated, so that the container 206 simply rests on the upper end 235 of the filling and injecting device 220. In a variation of this embodiment, there may be an interference fit between the container 206 and the walls of the cavity 246.

The filling and injecting device 220 preferably incorporates full radiation shielding around its side walls and one or more of its end walls, which is made of tungsten, tungsten plastic, lead and/or any other material that provides radiation protection from the radiopharmaceutical. Further, the syringe radiation shield 244 that surrounds the syringe 222 provides another level of shielding from radiopharmaceutical radiation. Thus, in the process of power filling the syringe 222 with the radiopharmaceutical or in the process of power injecting the radiopharmaceutical, persons handling the filling and injecting device 220 are shielded from radiopharmaceutical radiation; and the only potential for radiation leakage is through the centerhole 250. As mentioned earlier, nuclear medicine department personnel are disciplined in dealing with such a “live opening”, and such should not present a significant risk to radiation exposure.

As shown in FIG. 8, the filling and injecting device 220 may have a printed label 260. Further, the filling and injecting device 220 may have a radio frequency identification device (“RF-ID”) tag 262 that may be part of, or independent of, the label 260. As shown in FIG. 12, the microprocessor 245 may have an RF-ID interface 263 for reading data from and/or writing data to the RF-ID tag 262. The vial 202 may have an RF-ID tag 259, and/or the syringe 222 may have an RF-ID tag 261. An appropriate read/write device 255, which may be connected to a computer, may be used to read data from and/or write data to one or more of the RF-ID tags 259, 261, 262. The computer 257 may be located at any appropriate location and is shown as being located at the site of a user of the filling and injecting device 220 (e.g., a healthcare facility or pharmacy). Thus, data relating to the radiopharmaceutical, the syringe 222, the container 206 and/or filling and injecting operations can be read from and/or written to the RF-ID tag 262 with virtually every operation of the filling and injection device 220 (if desired), and such data may be available to the microprocessor 245 of the filling and injecting device 220. Further, data written to and/or read from one or more of the RF-ID tags 259, 260, 261 may be communicated (e.g., via the computer 257) to other computers, including remote computers in an appropriate manner (such as those known in the art).

Thus, upon deciding to utilize a particular radiopharmaceutical, data from the vial's label may be loaded into the microprocessor memory 247 one or both manually (e.g., via the user interface 254) and automatically (e.g., using the read/write device 255 and one or more of the RF-ID tags 259, 262). Data may be loaded into the syringe RF-ID tag 261 using the read/write device 255. Such data may include, but is not limited to, the following:
Prescription data.
Identification of the radiopharmaceutical, its brand, its supplier, etc.
Radioactivity level per mL as measured by a pharmacy.
Rate of radioactivity decay.
Calibration time and date.
The vial's usage history.
An expected remaining volume.
Expiration data.

A user can operate the user interface 254 to select portions of this data for display. Thus, prior to a filling operation, the control in the filling and injecting device 220 can be programmed to automatically or selectively check data including but not limited to
Efficacy of the expiration date and time.
Recall information.
Syringe installation and removal information to prevent reuse of a syringe.
Prior vial use and whether vial can be properly used now.
Efficacy of the fill program by checking the vial's expected remaining volume.
A calculation and display of the recommended fill volume, based on the pharmacy measured activity level, rate of decay data, the calibration time and date, and the prescribed dosage and injection time and date.
Product promotions from the radiopharmaceutical supplier.
Drug package insert information.

Data that may be manually programmed with the user interface 254 and/or written to the RF-ID tag 262 (e.g., via the read/write device 255) for use by the microprocessor 245 to at least generally control an operation of the filling and injection device 220 may include, but is not limited to, the following:
Fill volume of each fill.
The vial's remaining volume as calculated from usage history.
Date and time of each fill.
The radioactivity level for each fill.
Any other information to be entered by a user including but not limited to the following: Patient related information, device status, for example, service needs, usage history, etc.

The filling/injecting device 220 can consistently fill syringes with correct unit dose volumes to a very high accuracy in a single filling operation. This may eliminate the time-consuming and repetitive manual process of dose adjustment, and/or may reduce a user's risk of exposure to radiation. Thus, the wasteful and costly overfilling of syringes may be reduced or even eliminated, and/or the treatment provider may experience a more efficient use the pharmacy supplied vials.

The filling and injecting device 220 may monitor the backpressure generated during a power injection and may pause or terminate an injection that has an unusually high or low pressure. A low pressure may indicate an empty syringe or leak, and a high pressure may indicate a blockage or possible extravasation.

In an application where the filling and injecting device 220 is used by a pharmacy instead of the treatment provider to fill unit dose syringes, and an RF-ID tag is applied to the syringes, the filling and injecting device may be used to write some or all of the above-mentioned vial and syringe filling information to the syringe RF-ID tag.

In the exemplary embodiment of FIG. 9, the syringe mounting structure 228 is pivotable away from the body of the filling and injecting device 220. In alternative embodiments, syringe mounting structure may be completely separable from the filling and injecting device. Further, in the exemplary embodiments shown and described, the syringe 222 has a needle 234, however, the filling and injecting device 220 may be used to fill and/or dispense a radiopharmaceutical from a syringe that is not equipped with a needle. In such an embodiment, an intermediate connector may be utilized to interface with and provide a fluid interconnection with the vial 202. One example of an appropriate intermediate connector may include a needle on one end for penetrating a septum of the vial, and a fitting on an opposite end that is attachable to what may be characterized as a needle-free nozzle of the syringe (e.g., via an appropriate luer fitting). In addition, in FIG. 12, the filling and injecting device may be corded or cordless (e.g., battery powered).

In the exemplary embodiments shown and described, the filling and injecting device 220 is a hand-held device. However, the filling and injection device 220 may be either hand-held during a power injection of the radiopharmaceutical or it may be mounted to a support. Support mounted injections may, via an accessory cable or console, be remotely started, stopped and/or unattended after a manual start.

With regard to the illustrated embodiments, the radiation shields 204, 216 for the container 206 are described as being mounted in the cap 208 and the base 210, respectively. Other embodiments may include a radiation shield that may be fully or partially contained in the cap 208 and/or the base 210, or may be a separate and independent component(s) that is separately attachable to the cap 208 and/or base 210, or be of another appropriate configuration.

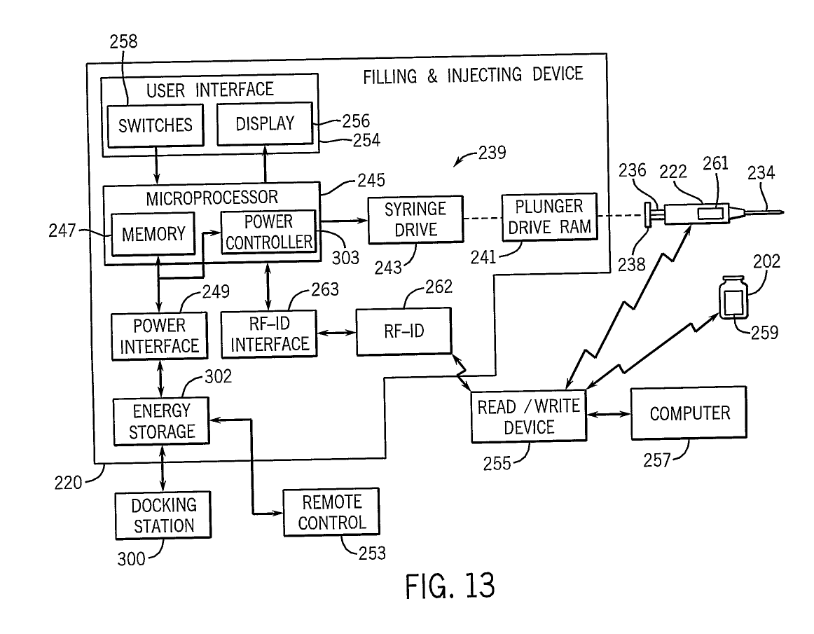

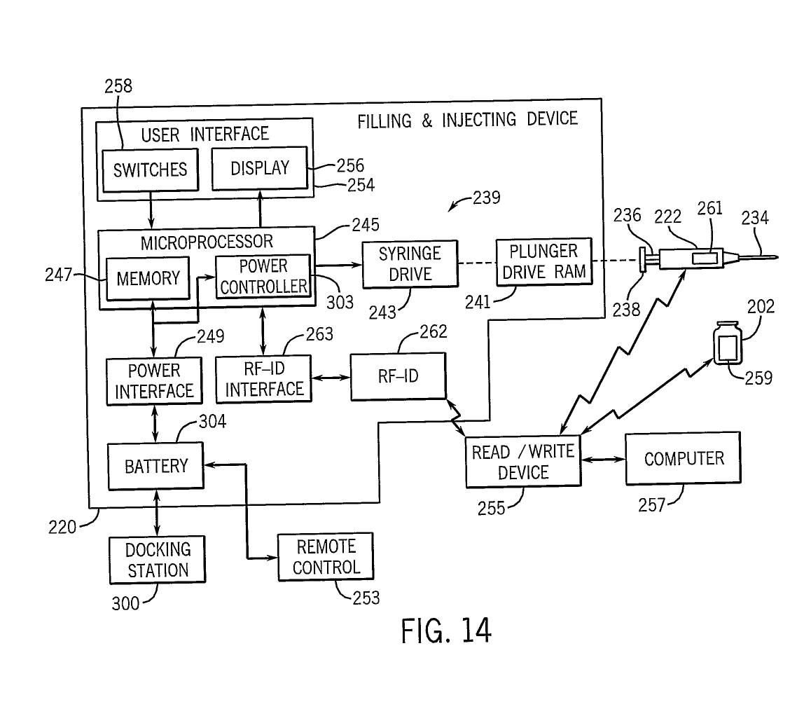

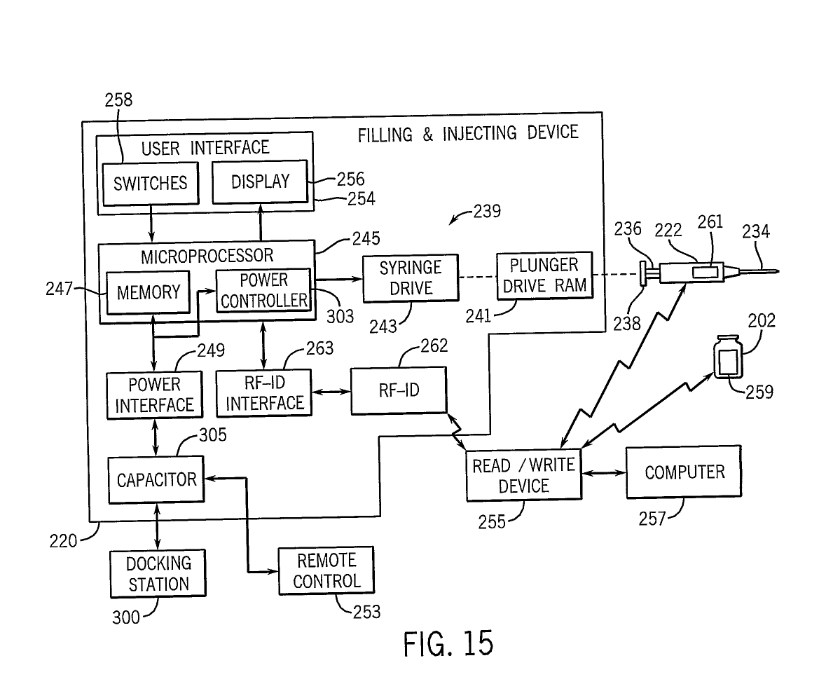

FIGS. 13-15 illustrate exemplary cordless filling and injecting devices 220. In the embodiment of FIG. 13, the cordless filling and injecting device 220 may feature an energy storage device 302, a docking station 300, and a power controller 303. Advantageously, the energy storage device 302 may support cordless operation of some embodiments of the filing and injecting device 220, as is described further below. The energy storage device 302 may include a battery 304, as illustrated by FIG. 14, or a capacitor 305, as illustrated by FIG. 15. The battery 304 may include a lead acid battery, a lithium ion battery, a lithium ion polymer battery, a nickel cadmium battery, a nickel-metal hydride battery, or an alkaline battery, for instance. The capacitor 305 may include an electrolytic capacitor, a tantalum capacitor, a super capacitor, a polyester film capacitor, a polypropylene capacitor, a polystyrene capacitor, a metallized polyester film capacitor, an epoxy capacitor, a ceramic capacitor, a multi-layered ceramic capacitor, a silver-mica capacitor, an adjustable capacitor, and/or an air core capacitor, for example. In other embodiments, the energy storage device 302 may include other forms of electrical energy storage, such as an inductor; mechanical energy storage, such as a pressurized fluid chamber, a flywheel or other kinetic energy storage device, a spring, and/or some other resilient member; and/or chemical energy storage, such as a fuel cell, for instance. The energy storage device 302 may be substantially or entirely non-ferrous in some embodiments adapted for use near a magnetic resonance imaging (MRI) machine.

As assembled, the docking station 300 may couple to the filling and injection device 220 and the energy storage device 302. The power controller 303 may be partially or entirely integrated into the microprocessor 245, or the power controller 303 may be independent of the microcontroller 245. The power controller 303 may communicate with the energy storage device 302 through the power interface 245. The power controller 303 may receive signals from the energy storage device 302 relating to various energy storage parameters, such as an energy storage level, temperature, a charging rate, or an energy discharge rate. For example, embodiments employing a capacitor 304 may also include protection circuitry to restrict the rate at which the capacitor charges and/or discharges, thereby limiting the exposure of other components to large currents. The protection circuitry may be partially or entirely integrated into the power controller 303 in some embodiments.

In operation, the power controller 303 may monitor and control the energy storage device 302. For instance, the power controller 303 may monitor and/or control a rate and/or level of charging of the energy storage device 302. Similarly, in some embodiments, the power controller 303 may monitor and/or control a rate and/or level of discharge of the energy storage device 303. For example, the power controller 303 may determine if the energy storage device 302 is charged to a pre-determined level, such as substantially charged or discharged, and transmit a signal to the display 256 and/or the docking station 300 indicative of the level.

In some embodiments, the power controller 303 and/or the microprocessor 245 may determine if the energy storage device 302 has an energy level sufficient to power a requested injecting or filing operation. If the energy storage device 302 has a sufficient energy level to power the operation, the power controller 303 and/or the microprocessor 245 may permit the operation. On the other hand, if the energy storage device 302 has an insufficient energy level to power the requested operation, the power controller 303 and/or the microprocessor 245 may transmit a warning signal, for instance to the display 256, and/or prevent the requested operation from proceeding.

The memory 247 and/or memory within the energy storage device 302, the power control 303, or other components of the filing and injecting device 220 may track the life cycle of the energy storage device 302. For example, the number of times the energy storage device 302 has been charged and/or discharged may be counted and retained by memory. In some embodiments, the microprocessor 245 and/or the power controller 303 may transmit a signal to the display 256 indicative of the life of the energy storage device 302. For instance, an end-of-life warning signal and/or charge/discharge cycle count may be transmitted to and displayed by the display 256. In some embodiments, the energy storage device 302 may include memory for storing information indicative of its life cycle, such as a date of manufacturing, a tracking number, a charge/discharge cycle count, an energy storage device type, a manufacture identifier, an expiration date, and/or a remaining storage capacity, for example. Additionally, in some embodiments, the energy storage device 302 may include RFID circuitry for communicating with other devices.

In some embodiments, the docking station 300 may energize the energy storage device 302. Alternatively, or additionally, the energy storage device 302 may receive energy from sources other than the docking station 300, such as energy from a photoelectric device, a hand crank or other manual energizing device, and/or an energy scavenging device coupled to the filing and injecting device 220.

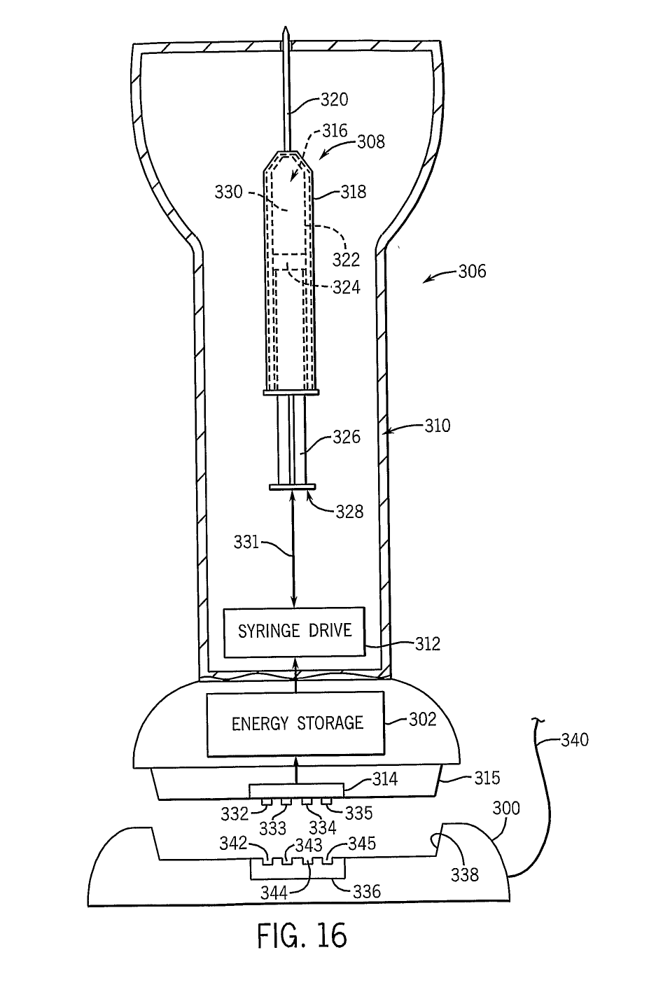

FIG. 16 illustrates an exemplary cordless filling and injecting device 306 having an energy storage device 302 and couplable to a docking station 300. The exemplary cordless filling and injecting device 306 may include features of the previously discussed filing and injecting devices. The present cordless filling and injecting device may feature a shielded syringe assembly 308, shielding 310, a syringe drive 312, a docking station electrical interface 314, and a docking station mechanical interface 315. The docking station electrical interface 314 may include a plurality of leads 332, 333, 334, 335. In the present embodiment, syringe assembly 308 may include a syringe 316 and shielding 318. The illustrated syringe 316 may have a needle 320, a barrel 322, a plunger 324, and a plunger rod 326 having an outer end 328. One or more fluids 330 may be disposed within the barrel 322 of the syringe 316. For example, the fluid 330 may include a radiopharmaceutical, a contrast agent, saline, a tagging agent, or other pharmaceuticals, for instance. In some embodiments, the syringe 316 may be a single stage syringe, a two stage syringe with different fluids in each stage, a multi-barrel syringe, or a syringe having more than two stages and more than two fluids.

The shielding 310, 318 may include electromagnetic shielding, radiation shielding, thermal shielding, or some combination thereof. In some embodiments, the shielding 310, 318 may feature radiation shielding materials, such as lead, depleted uranium, tungsten, tungsten impregnated plastic, etc. Alternatively, or additionally, shielding 310, 318 may include electromagnetic shielding materials, such as a layer, mesh, or other form of copper, steel, conductive plastic, or other conductive materials. In certain embodiments, the shielding 310, 318 is substantially or entirely non-ferrous. The shielding 310 may entirely envelope the syringe 316, the syringe drive 312, and/or the energy storage device 302; substantially envelope one or more of these components 316, 312, 302; or partially envelope one or more of these components 316, 312, 302. Similarly, the shielding 318 may entirely, substantially, or partially envelope the syringe 316. It should also be noted that some embodiments may not include shielding 310 and/or 318, which is not to suggest that any other feature discussed herein may not also be omitted.

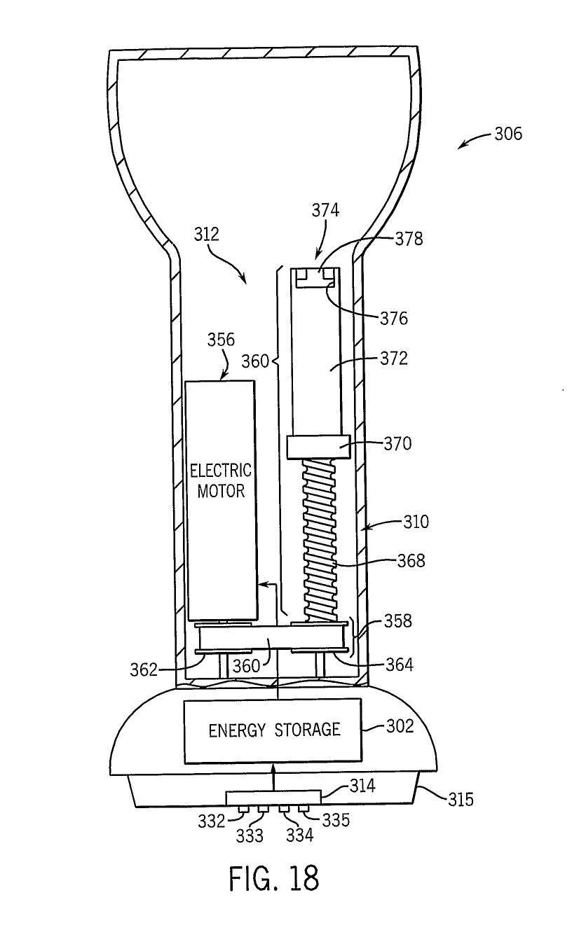

The syringe drive 312 may include a piezoelectric drive, a linear motor, a shape memory alloy, a rack-and-pinion system, a worm gear and wheel assembly, a planetary gear assembly, a belt drive, a gear drive, a manual drive, and/or a pneumatic drive. For example, in the embodiment of FIG. 18, discussed below, the syringe drive 312 may include an electric motor and a screw drive. In some embodiments, the syringe drive 312 may be entirely, substantially, or partially non-ferrous.

The docking station 300 may include a complimentary electrical interface 336, a complimentary mechanical interface 338, and a power cable 340. The complimentary electrical interface 336 may include a plurality of female connectors 342, 343, 344, 345. The power cable 340 may be adapted to receive power from a wall outlet, and the docking station 300 may include power conditioning circuitry, such as a transformer, rectifier, and low-pass power filter. In some embodiments, the docking station may be configured to accept wall-outlet AC power and output DC power via female connectors 342, 343, 344, 345. In certain embodiments, the docking station 300 may include an independent power source, such as a battery, or a generator. For example, the generator may include solar cells, a gas motor powered generator, a mechanical crank coupled to a generator, and so forth. Moreover, the docking station 300 may be mounted on a movable stand, a rotatable arm, a car, an imaging device, a patient table, a wall mount, or another suitable mount.

In operation, the cordless filling and injection device 306 may mate with the docking station 300. Specifically, the docking station mechanical interface 315 may mate with the complimentary mechanical interface 338 and the docking station electrical interface 314 may mate with the complimentary electric interface 336. Power may flow through the power cable 340 through the female connectors 342, 343, 344, 345 and into the male connectors 332, 333, 334, 335. Power may flow into the energy storage device 302. In some embodiments, the energy storage device 302 may be charged while the syringe 316 is being filled. For instance, while the energy storage device 302 is charging, the syringe drive 312 may apply force 331 that moves the plunger 324 down within the barrel 322, thereby tending to draw a fluid into the barrel 322. During filing, in situ or ex situ feed-forward or feed-back control may be exercised over the fill rate and/or fill volume.

When the energy storage device 302 is charged or energized, the cordless filling and injecting device 306 maybe removed from the docking station 300 and used to inject a radio pharmaceutical 330, tagging agent, or other substance without any power cables interfering with the procedure. Injection may be performed at the same site at which the cordless filling and injecting device 306 is filled and charged, or the cordless filling and injecting device 306 may be shipped in a charged and filled state for use at another site. During injection, energy may flow from the energy storage device 302 to the syringe drive 312, which may apply force 331 to the outer end 328 of the push rod 326. The plunger rod 326 may drive plunger 324 through the barrel 332 and inject fluid 330. During injection, in situ or ex situ feed-forward or feed-back control may be exercised over the rate and/or volume of injection.

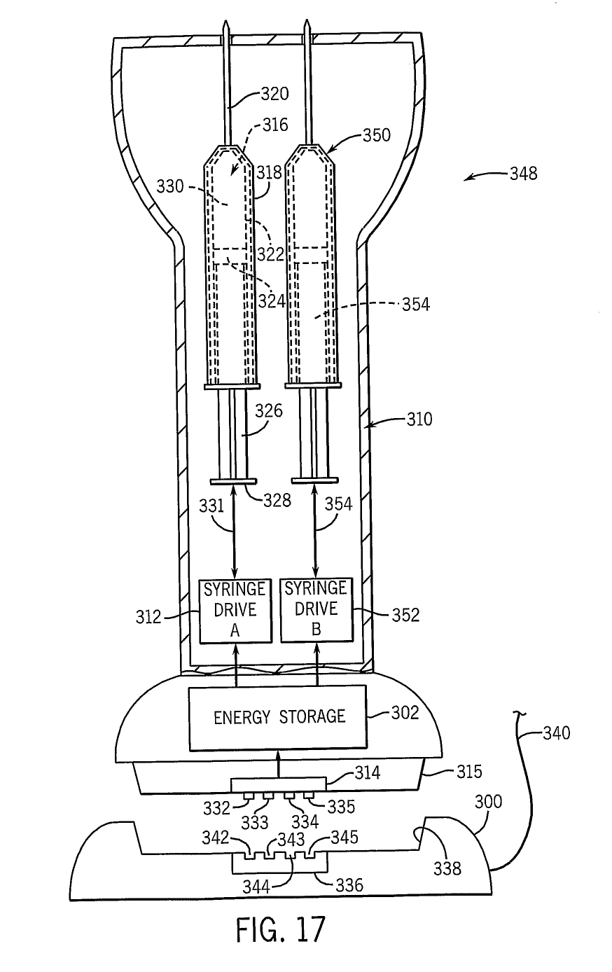

FIG. 17 illustrates an exemplary cordless filling and injecting device having dual syringes 348. The present cordless filling and injecting device 348 may include a secondary syringe 350 and a secondary syringe drive 352. The secondary syringe 350 may be shielded and may include fluid 354, which may be one or more of the previously listed fluids 330. In the present embodiment, the secondary syringe 350 may be within shielding 310, but in other embodiments the secondary syringe 350 may be partially or entirely external to shielding 310. In addition, the dual syringes 348 may be independent from one another or an integral or united multi-barrel syringe.

In operation, the syringe drive 352 may apply a force 354 to the secondary syringe 350 and drive fluid 354 out of the secondary syringe 350 or into the secondary syringe 350. In some embodiments, syringe drive 312 and secondary syringe drive 352 may be partially or entirely integrated into a single syringe drive. Alternatively, syringe drive 312 and secondary syringe drive 352 may be independent syringe drives. During injecting and/or filing, independent, in situ or ex situ feed-forward or feed-back control over the flow rate and/or volume of fluids 330 and/or 354 injected or filled by the cordless filling and injecting device 348 may be exercised.

FIG. 18 illustrates an exemplary syringe drive 312 within the cordless filling and injecting device 306. The illustrated syringe drive 312 may include an electric motor 356, a transmission 358, and a linear drive 360. The electric motor 356 may be a DC electric motor or an AC electric motor, such as a stepper motor. The illustrated transmission 358 may include a primary pulley 362, a secondary pulley 364, and a belt 366. The present linear drive 360 may have a externally threaded shaft, worm, or screw 368, a bushing 370, an outer shaft 372, and a syringe interface 374. The transmission 358 may be a reducing transmission. For example, the ratio of the diameter of the secondary pulley 364 to the diameter of the primary pulley 362 may be greater than 1.5:1, 2:1, 3:1, 4:1, 5:1, 8:1, 12:1, or more. The syringe interface 374 may include a wider, outer-end receptacle 376 and a shaft slot 378. In some embodiments, some or all of these components 356, 358, 360 may be substantially or entirely non-ferrous. Further, some or all of these components 356, 358, 360 may be partially, substantially, or entirely shielded by shielding 310.

In operation, the electric motor 356 may drive the primary pulley 362. As the primary pulley 362 rotates, the belt 366 may rotate the secondary pulley 364. The rotation of the secondary pulley 364 may drive the screw 368, which may rotate within the bushing 370. The bushing 370 may be threaded so that rotation of the screw 368 applies a linear force to the bushing 370. A linear sliding mechanism may prevent rotation of the bushing 370 while permitting the bushing 370 to translate up and down the screw 368. As the screw 368 rotates, the outer shaft 372 may be pulled down the screw 368 or pushed up the screw 368 by the bushing 370. The outer shaft 372 may linearly translate relative to the screw 368 and drive the syringe 316 via the syringe interface 374.

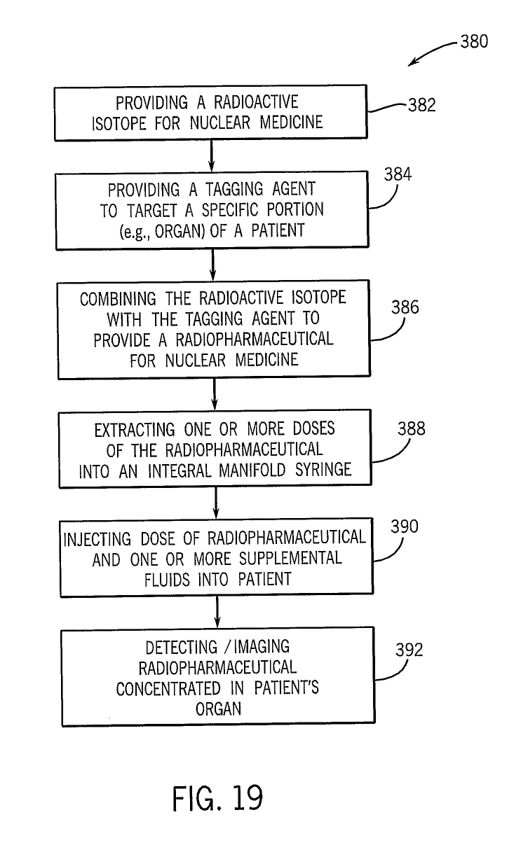

FIG. 19 is a flowchart illustrating an exemplary nuclear medicine process utilizing one or more syringes as illustrated with reference to FIGS. 1-18. As illustrated, the process 380 begins by providing a radioactive isotope for nuclear medicine at block 382. For example, block 382 may include eluting technetium-99m from a radioisotope generator. At block 384, the process 380 proceeds by providing a tagging agent (e.g., an epitope or other appropriate biological directing moiety) adapted to target the radioisotope for a specific portion, e.g., an organ, of a patient. At block 386, the process 380 then proceeds by combining the radioactive isotope with the tagging agent to provide a radiopharmaceutical for nuclear medicine. In certain embodiments, the radioactive isotope may have natural tendencies to concentrate toward a particular organ or tissue and, thus, the radioactive isotope may be characterized as a radiopharmaceutical without adding any supplemental tagging agent. At block 388, the process 380 then may proceed by extracting one or more doses of the radiopharmaceutical into a syringe or another container, such as a container suitable for administering the radiopharmaceutical to a patient in a nuclear medicine facility or hospital. At block 390, the process 380 proceeds by injecting or generally administering a dose of the radiopharmaceutical and one or more supplemental fluids into a patient. After a pre-selected time, the process 380 proceeds by detecting/imaging the radiopharmaceutical tagged to the patient's organ or tissue (block 392). For example, block 392 may include using a gamma camera or other radiographic imaging device to detect the radiopharmaceutical disposed on or in or bound to tissue of a brain, a heart, a liver, a tumor, a cancerous tissue, or various other organs or diseased tissue.

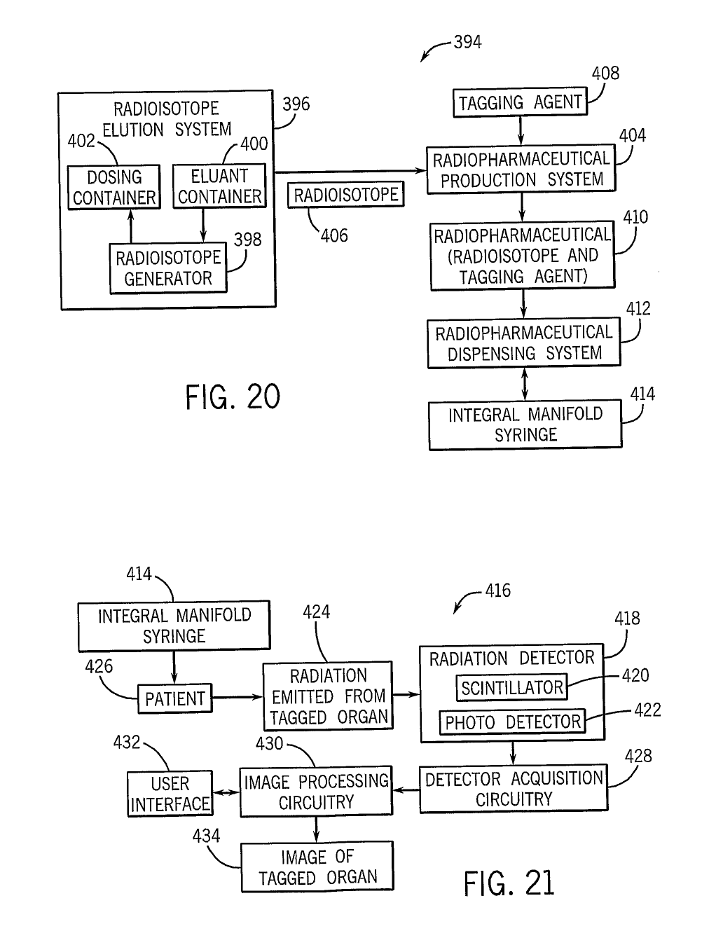

FIG. 20 is a block diagram of an exemplary system 394 for providing a syringe having a radiopharmaceutical disposed therein for use in a nuclear medicine application. For example, the syringe may be one of the syringes illustrated and described with references to FIGS. 1-18. As illustrated, the system 394 may include a radioisotope elution system 396 having a radioisotope generator 398, an eluant supply container 400, and an eluate output container or dosing container 402. In certain embodiments, the eluate output container 402 may be in vacuum, such that the pressure differential between the eluant supply container 400 and the eluate output container 402 facilitates circulation of an eluant (e.g., saline) through the radioisotope generator 398 and out through an eluate conduit into the eluate output container 402. As the eluant, e.g., a saline solution, circulates through the radioisotope generator 398, the circulating eluant generally washes out or elutes a radioisotope, e.g., Technetium-99m. For example, one embodiment of the radioisotope generator 398 may include a radiation shielded outer casing (e.g., lead shell) that encloses a radioactive parent, such as molybdenum-99, adsorbed to the surfaces of beads of alumina or a resin exchange column. Inside the radioisotope generator 398, the parent molybdenum-99 transforms, with a half-life of about 67 hours, into metastable technetium-99m. The daughter radioisotope, e.g., technetium-99m, is generally held less tightly than the parent radioisotope, e.g., molybdenum-99, within the radioisotope generator 398. Accordingly, the daughter radioisotope, e.g., technetium-99m, can be extracted or washed out with a suitable eluant, such as an oxidant-free physiologic saline solution. The eluate output from the radioisotope generator 398 into the eluate output container 402 generally includes the eluant and the washed out or eluted radioisotope from within the radioisotope generator 398. Upon receiving the desired amount of eluate within the eluate container 402, a valve may be closed to stop the eluant circulation and output of eluate. As discussed in further detail below, the extracted daughter radioisotope can then, if desired, be combined with a tagging agent to facilitate diagnosis or treatment of a patient (e.g., in a nuclear medicine facility).

As further illustrated in FIG. 20, the system 394 also may include a radiopharmaceutical production system 404, which functions to combine a radioisotope 406 (e.g., technetium-99m solution acquired through use of the radioisotope elution system 396) with a tagging agent 408. In some embodiments, this radiopharmaceutical production system 404 may refer to or include what are known in the art as “kits” (e.g., Technescan® kit for preparation of a diagnostic radiopharmaceutical). Again, the tagging agent may include a variety of substances that are attracted to or targeted for a particular portion (e.g., organ, tissue, tumor, cancer, etc.) of the patient. As a result, the radiopharmaceutical production system 404 produces or may be utilized to produce a radiopharmaceutical including the radioisotope 406 and the tagging agent 408, as indicated by block 410. The illustrated system 394 may also include a radiopharmaceutical dispensing system 412, which facilitates extraction of the radiopharmaceutical into a vial or syringe 414 as illustrated in FIGS. 1-18. In certain embodiments, the various components and functions of the system 814 may be disposed within a radiopharmacy, which prepares the syringe 414 of the radiopharmaceutical for use in a nuclear medicine application. For example, the syringe 414 may be prepared and delivered to a medical facility for use in diagnosis or treatment of a patient.

FIG. 21 is a block diagram of an exemplary nuclear medicine imaging system 416 utilizing the syringe 414 of radiopharmaceutical provided using the system 394 of FIG. 20. As illustrated, the nuclear medicine imagining system 416 may include a radiation detector 418 having a scintillator 420 and a photo detector 422. In response to radiation 428 emitted from a tagged organ within a patient 426, the scintillator 420 emits light that may be sensed and converted to electronic signals by the photo detector 422. Although not illustrated, the imaging system 416 also can include a collimator to collimate the radiation 424 directed toward the radiation detector 418. The illustrated imaging system 416 also may include detector acquisition circuitry 428 and image processing circuitry 430. The detector acquisition circuitry 428 generally controls the acquisition of electronic signals from the radiation detector 418. The image processing circuitry 430 may be employed to process the electronic signals, execute examination protocols, and so forth. The illustrated imaging system 416 also may include a user interface 432 to facilitate user interaction with the image processing circuitry 430 and other components of the imaging system 416. As a result, the imaging system 416 produces an image 434 of the tagged organ within the patient 426.

When introducing elements of various embodiments of the present invention, the articles “a”, “an”, “the”, and “said” are intended to mean that there are one or more of the elements. The terms “comprising”, “including”, and “having” are intended to be inclusive and mean that there may be additional elements other than the listed elements. Moreover, the use of “top”, “bottom”, “above”, “below” and variations of these terms is made for convenience, but does not require any particular orientation of the components.

While the invention may be susceptible to various modifications and alternative forms, specific embodiments have been shown by way of example in the figures and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A radiopharmaceutical apparatus comprising:
a first computer configured to transmit data about a radiopharmaceutical;
a radiopharmaceutical pig comprising:
radiation-shielding material;
a second computer configured to receive the data about a radiopharmaceutical from the first computer and calculate a dosage of a radiopharmaceutical based on the data received from the first computer; and
a first electrical connector mounted on the radiopharmaceutical pig and electrically connected to the second computer; and
a second electrical connector having a wired connection to the first computer, the second electrical connector being connectable with the first electrical connector to electrically connect the first computer with the second computer.

2. The apparatus of claim 1 further comprising a base unit supporting the first electrical connector.

3. The apparatus of claim 1 further comprising a control unit supporting the first computer.

4. The apparatus of claim 3 wherein the control unit further supports the first electrical connector.

5. The apparatus of claim 3 further comprising a dose calibrator electrically connected to and controlled by the control unit.

6. A radiopharmaceutical apparatus comprising:
a first computer;
a radiopharmaceutical pig comprising:
radiation-shielding material;
a second computer configured to:
receive a signal from the first computer that indicates when a radiopharmaceutical was measured; and
calculate an elapsed time since the radiopharmaceutical was measured; and
a first electrical connector mounted on the radiopharmaceutical pig and electrically connected to the second computer;
a base unit configured to mechanically support the radiopharmaceutical pig; and
a second electrical connector on the base unit, the second electrical connector having a wireless connection to the first computer, and being connectable with the first electrical connector to electrically connect the second computer with the first computer.

7. The apparatus of claim 6 further comprising a control unit supporting the second computer.

8. The apparatus of claim 7 further comprising a dose calibrator electrically connected to and controlled by the control unit.

9. A radiopharmaceutical pig comprising:
a body comprising radiation-shielding material and having a receptacle defined therein that is adapted to accommodate a radiopharmaceutical container;
a lid comprising radiation-shielding material, the lid being releasably attachable to the body to enable a radiopharmaceutical container to be enclosed within the pig; and
a computer comprising a memory and an input/output device, the computer being a component of one of the body and the lid, wherein the computer is configured to:
receive data indicative of radioactivity of a radiopharmaceutical;
receive data indicative of a time at which the radioactivity was measured;
calculate an elapsed time since the radioactivity was measured; and
calculate a volumetric dosage of the radiopharmaceutical based on the data received; and
an electrical connector electrically connected to the computer.

10. The radiopharmaceutical pig of claim 9 further comprising a first electrical connector electrically connected to the computer and mounted on an external surface of one of the body and the lid.

11. The radiopharmaceutical pig of claim 9 further comprising a first electrical connector electrically connected to the computer and mounted on an external surface of one of the body and the lid, wherein the first electrical connector is mounted on an end surface of the lid.

12. The radiopharmaceutical pig of claim 9 further comprising a first electrical connector electrically connected to the computer and mounted on an external surface of one of the body and the lid, wherein the first electrical connector is mounted on an end surface of the body and the input/output device is mounted on the body.

13. The radiopharmaceutical pig of claim 9 further comprising a base unit, a first electrical connector electrically connected to the computer and mounted on an external surface of one of the body and the lid, and a second electrical connector supported by the base unit, the second electrical connector being connectable with the first electrical connector.

14-81. (canceled)

82. The apparatus of claim 1 wherein the first computer and the second electrical connector are integrated into a control unit.

83. The apparatus of claim 1 comprising a RFID tag coupled to the radiopharmaceutical pig.

84. The apparatus of claim 1 wherein the data comprises the radioactivity level of the radiopharmaceutical.

85. The apparatus of claim 1 wherein the first computer is configured to receive data.

86. The apparatus of claim 85 wherein the data comprises information related to the radiopharmaceutical.

87. The apparatus of claim 6 wherein the signal comprises an RF signal.

88. The radiopharmaceutical pig of claim 10 wherein the first electrical connector is mounted on an end surface of the body.
